(19) United States
(12) Patent Application Publication
(10) Pub. No.: US 2008/0177091 A1
(43) Pub. Date: Jul. 24, 2008

(54) METHODS FOR PRODUCING AND PURIFYING PHENOLPHTHALEIN

(75) Inventors: Rajshekhar Madhukar Basale, Bangalore (IN); Balakrishnan Ganesan, Bangalore (IN); Ashok S Shyadligeri, Bangalore (IN); Veeraraghavan Srinivasan, Bangalore (IN)

Correspondence Address: CANTOR COLBURN LLP - SABIC (LEXAN/CYCOLOY), 20 Church Street, 22nd Floor, Hartford, CT, 06103, US

(73) Assignee: GENERAL ELECTRIC COMPANY, Schnectady, NY (US)

(21) Appl. No.: 11/626,671

(22) Filed: Jan. 24, 2007

(51) Int. Cl. C07D 307/83

(52) U.S. Cl. 549/308

(57) ABSTRACT

A phthalic anhydride, a phenol, a catalyst, and a promoter are reacted to form a reaction mixture comprising phenolphthalein. The reaction mixture is treated with a first solvent system to form a slurry. The first solvent system comprises a first polar organic solvent. In some embodiments the first solvent system additionally comprises a non-polar organic solvent. Use of the solvent system to treat the reaction mixture simplifies subsequent purification.

### BACKGROUND

Phenolphthalein is useful as a starting material to make a wide range of products. Producing phenolphthalein on a commercial scale with high purity has been a challenge. Presently available manufacturing processes for phenolphthalein are time consuming and require large amounts of energy, unit operations, chemicals, and equipment. In addition, some of the materials produced during the purification process are very viscous and difficult to handle. Thus, there remains a need for producing purified phenolphthalein on a commercial scale which requires fewer resources, less time, less equipment, or a combination thereof.

### BRIEF SUMMARY

Some or all of the above-described deficiencies are addressed by a method of producing a phenolphthalein comprising reacting a phthalic anhydride and a phenol in the presence of a catalyst and a promoter to form a reaction mixture comprising phenolphthalein; treating the reaction mixture with a first solvent system to form a slurry, wherein the first solvent system comprises a first polar organic solvent; filtering the slurry to obtain a first solid material; washing the first solid material with water to obtain a second solid material, wherein the water is at a temperature of 25° C. to 90° C.; wherein the second solid material comprises a phenolphthalein of formula (I)

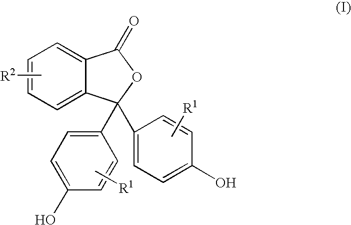

wherein R¹ is independently selected from the group consisting of a hydrogen and a hydrocarbyl group; R² is selected from the group consisting of a hydrogen, a hydrocarbyl group, and a halogen; and wherein the second solid material comprises greater than or equal to 97 weight percent of phenolphthalein based on the total weight of the second solid material.

In another embodiment, a method of producing a phenolphthalein, comprises reacting a phthalic anhydride and a phenol in the presence of a metal halogenate catalyst and an acid promoter to form a reaction mixture comprising phenolphthalein; treating the reaction mixture with a first solvent system to form a slurry, wherein the first solvent system comprises a polar and non-polar solvent; filtering the slurry to obtain a first solid material; washing the first solid material with water to obtain a second solid material, wherein the water is at a temperature of 25° C. to 90° C.; wherein the second solid material comprises a phenolphthalein of formula (I), and wherein the second solid material comprises greater than or equal to 97 weight percent of phenolphthalein based on the total weight of the second solid material.

In yet another embodiment, a method of purifying a phenolphthalein comprises treating a crude phenolphthalein material with a first solvent system to form a slurry, wherein the first solvent system comprises a first polar organic solvent; filtering the slurry to obtain a first solid material; and washing the first solid material with water to obtain a second solid material, wherein the water is at a temperature of 25° C. to 90° C.; wherein the second solid material comprises a phenolphthalein of formula (I), and wherein the second solid material comprises greater than or equal to 97 wt % of phenolphthalein based on the total weight of the second solid material.

In still another embodiment, a method of producing a phenolphthalein comprises reacting a phthalic anhydride and a phenol in the presence of zinc chloride and chloro sulphonic acid to form a reaction mixture comprising phenolphthalein; treating the reaction mixture with a first solvent system to form a slurry, wherein the first solvent system comprises a methanol and toluene; filtering the slurry to obtain a first solid material; washing the first solid material with water to obtain a second solid material, wherein the water is at a temperature of 25° C. to 90° C.; wherein the second solid material comprises a phenolphthalein of formula (I), and wherein the second solid material comprises greater than or equal to 97 weight percent of phenolphthalein based on the total weight of the second solid material.

### DETAILED DESCRIPTION

It has now been discovered that phenolphthalein can be obtained by processes comprising treating a reaction mixture comprising crude phenolphthalein with a polar organic solvent or a mixture of a polar organic solvent and a non-polar organic solvent. The new processes provide easy handling of the reaction mixtures after the completion of the reaction. The new processes also help remove undesired impurities in a single-pot operation. Thus the new processes are especially commercially attractive as they can simplify purification steps and reduce manufacturing costs.

Phenolphthalein can be prepared by a condensation reaction between a phthalic anhydride and a phenol in the presence of a catalyst and a promoter.

The phenol may be a substituted or unsubstituted phenol of formula (II):

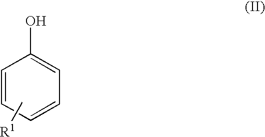

wherein R¹ is selected from the group consisting of a hydrogen and a hydrocarbyl group. As used herein, the term “hydrocarbyl” is defined as a monovalent moiety formed by removing a hydrogen atom from a hydrocarbon. Representative hydrocarbyls are alkyl groups having 1 to 25 carbon atoms, such as methyl, ethyl, propyl, butyl, pentyl, hexyl, heptyl, octyl, nonyl, undecyl, decyl, dodecyl, octadecyl, nonadecyl, eicosyl, heneicosyl, docosyl, tricosyl, and the isomeric forms thereof; aryl groups having 6 to 25 carbon atoms, such as ring-substituted and ring-unsubstituted forms of phenyl, tolyl, xylyl, naphthyl, biphenyl, tetraphenyl, and the like; aralkyl groups having 7 to 25 carbon atoms, such as ring-substituted and ring-unsubstituted forms of benzyl, phenethyl, phenpropyl, phenbutyl, naphthoctyl, and the like; and cycloalkyl groups, such as cyclopropyl, cyclobutyl, cyclopentyl, cyclohexyl, cycloheptyl, cyclooctyl, and the like. The term “aryl” as used herein refers to various forms of aryl groups that have been described hereinabove for the “hydrocarbyl” group. In one embodiment, the R¹ is a hydrogen.

The amount of the reactant phenol can be 60 to 90 mole percent (mol %), or more specifically, 70 to 85 mol %, based on the total moles of phenol and phthalic anhydride.

The phthalic anhydride can be a substituted or unsubstituted phthalic anhydride of formula (III):

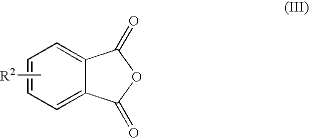

wherein R² is selected from the group consisting of a hydrogen, a hydrocarbyl group, and a halogen. The term “hydrocarbyl” is as defined above. In one embodiment, the R² is a hydrogen.

The amount of phthalic anhydride can be 10 to 40 mol %, or more specifically, 15 to 30 mol %, based on the total moles of phenol and phthalic anhydride. In some embodiments the phthalic anhydride can be used as the limiting reactant in the reaction. In such cases, the molar ratio of phthalic anhydride to phenol in the starting materials is less than 1:2.

A catalyst is generally used to facilitate the formation of phenolphthalein. Examples of suitable catalysts include a metal halogenate, an inorganic acid, thionyl chloride, sulphuryl chloride, and combinations of two or more of the foregoing. Exemplary metal halogenate catalysts include zinc chloride, aluminum chloride, stannic chloride, and combinations of two or more of the foregoing. Exemplary inorganic acids include hydrochloric acid, sulphuric acid, methanesulfonic acid, and combinations of two or more of the foregoing.

The catalyst can be present in the amount of 3 to 20 mol %, or more specifically, 5 to 14 mol %, based on the total moles of reactants, catalyst, and promoter. In one embodiment, the catalyst is zinc chloride. In some embodiments, the zinc chloride is present in an amount of 10 to 20 mol % based on the total moles of reactants, catalyst, and promoter.

A promoter can be used to promote the formation of phenolphthalein. Suitable promoters include chloro sulphonic acid, trichloro acetic acid, methanesulfonic acid, dodecylbenzenesulfonic acid, triflic acid, boron trifluoride, p-toluene sulphonyl chloride, and combinations of two or more of the foregoing. In one embodiment, the promoter is chloro sulphonic acid.

The promoter can be present in the amount of 1 to 6 mol %, or more specifically, 2 to 5 mol %, based on the total moles of reactants, catalyst, and promoter.

The reaction of the phenol and phthalic anhydride can be conducted at a temperature of 75° C. to 150° C., or more specifically, 80° C. to 140° C. at atmospheric pressure. The reaction time can be 3 to 40 hours, or more specifically, 5 to 30 hours, or even more specifically, 8 to 20 hours.

In one embodiment the reaction mixture is treated with a first solvent system to form a slurry. The reaction mixture can be mixed with the first solvent system at a temperature of 20 to 50° C., or, more specifically, 25 to 40° C., for 1 to 5 hours, or, more specifically, 2 to 3 hours. In some embodiments the slurry is refluxed for a period of time of 10 minutes to 2 hours. In one embodiment, the first solvent system consists essentially of a polar organic solvent selected from the group consisting of methanol, ethanol, isopropanol, propanol, chloroform, acetone, ethyl acetate, phenol, and combinations of two or more of the foregoing.

In another embodiment, the first solvent system comprises a polar organic solvent. Suitable polar organic solvents include methanol, ethanol, isopropanol, propanol, chloroform, acetone, ethyl acetate, phenol, and combinations of two or more of the foregoing.

In some embodiments, the first solvent system further comprises a non-polar organic solvent. In these embodiments, the first solvent system comprises a mixture of a polar organic solvent and a non-polar organic solvent. Suitable non-polar organic solvents include aromatic hydrocarbons having 6 to 14 carbons, aliphatic hydrocarbons having 5 to 8 carbons, non-polar chlorinated hydrocarbons and combinations of two or more of the foregoing. Non-limiting examples of suitable aromatic hydrocarbon solvents include toluene, xylene, cumene, benzene and the like. Non-limiting examples of suitable aliphatic hydrocarbon solvents include hexane, cyclohexane, pentane, and the like. Non-limiting examples of non-polar chlorinated hydrocarbon solvents include 1,2-dichloroethane and the like.

Non-limiting examples of suitable polar organic solvent and non-polar organic solvent mixtures include methanol:toluene, ethyl acetate:toluene, methanol:hexane, ethyl acetate: 1,2-dichloroethane, acetone: 1,2-dichloroethane, acetone:toluene, acetone:hexane, and isopropanol:toluene.

The volume ratio (v:v) of the polar organic solvent to the non-polar organic solvent in the first solvent system can be 2:98 to 50:50. In one embodiment, the polar organic solvent is methanol and the non-polar organic solvent is toluene in a volume ratio of 2:98 to 25:75.

Treating the reaction mixture with the first solvent system can greatly minimize the number of additional purification steps needed to obtain a purified phenolphthalein, which may result in an improvement of the handling of the reaction mixtures, better yield, reduction in production costs, or a combination of some or all of these advantages.

When treating is completed, the slurry is filtered to obtain a first solid material. The filtered first solid material may be washed with water having a temperature of 25° C. to 90° C., or more specifically, 50° C. to 90° C. The first solid material may be washed one or more times. After the water wash, a second solid material is isolated. Suitable isolation methods include filtration, decanting and other suitable methods known in the art. The second solid material comprises greater than or equal to 97 wt %, or more specifically, greater than or equal to 98 wt %, of phenolphthalein, based on the total weight of the second solid material.

The second solid material can be dissolved in a second solvent system, optionally at an elevated temperature as described below, to obtain a first solution for further purification. In one embodiment, the second solvent system comprises a second polar organic solvent such as an aliphatic alcohol. Non-limiting examples of aliphatic alcohols include methanol, ethanol, iso-propanol, iso-butanol, n-butanol, tertiary butanol, n-pentanol, iso-pentanol, cyclohexanol, ethylene glycol, propylene glycol, neopentyl glycol and the like. Methanol is especially useful.

In some embodiments, the first solution is treated with an adsorbent such as activated carbon or other decolorizing agent to obtain a first treatment mixture to remove residual colored impurities. Typically, the adsorbent is mixed with the first solution for 0.1 to 2 hours at the temperature of dissolution. Then, the first treatment mixture is filtered to obtain a second solution. The resulting second solution is then subjected to crystallization to obtain a third solid material. Once the third solid material has crystallized from the second solution, the third solid material is isolated and dried. Suitable isolation methods include filtration, decanting and other suitable methods known in the art.

In some embodiments the second solid material is dissolved in a second solvent system at an elevated temperature (but below the decomposition temperature of phenolphthalein) to form a solution. The solution may optionally be treated with a decolorizing agent as described above. Then, a portion of the solvent is removed to get a supersaturated solution. As the supersaturated solution cools the third solid material crystallizes from the solution. The third solid material is isolated under ambient conditions and then dried. Suitable isolation methods include filtration, decanting and other suitable methods known in the art. In some embodiments, the third solid material comprises greater than or equal to 98 wt % of phenolphthalein, based on the total weight of the third solid material.

In one embodiment, the third solid material comprises greater than or equal to 98 wt %, or more specifically, 99 wt %, or even more specifically, 99.6 wt %, of phenolphthalein, based on the total weight of the third solid material.

The above-described methods have phenolphthalein molar yield of greater than or equal to 60%, or, more specifically, greater than or equal to 70%, or, even more specifically, greater than or equal to 80%, based the molar amount of phthalic anhydride in the starting materials.

As described above, the phenolphthalein may be a substituted or unsubstituted phenolphthalein of formula (I):

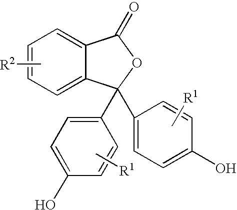

wherein R¹ is independently selected from the group consisting of a hydrogen and a hydrocarbyl group, and R² is selected from the group consisting of a hydrogen, a hydrocarbyl group, and a halogen.

Additionally, crude phenolphthalein materials can be treated with the first solvent system and any combination of the subsequent steps described above to achieve an isolated material have a phenolphthalein content of greater than or equal to 97 wt %.

The above-described processes are further illustrated by the following non-limiting examples.

### EXAMPLES

HPLC analysis was generally carried out by using a solution of about 50 milligrams of the sample dissolved in about 10 milliliters of acetonitrile:0.02% aqueous H₃PO₄ (70:30, v:v). The HPLC instrument was equipped with a C18 (reverse phase) column maintained at a temperature of 40° C., and an ultraviolet detector capable of detecting components at a wavelength of 225 nanometers. The flow rate was maintained at 1 milliliter per minute. Area percent assay was computed from the area value for each peak detected in the chromatogram divided by the total area from all peaks detected. To measure weight percent assay, calibration curves for phenol, phthalic anhydride, and phenolphthalein were first generated. Then the weight percent of a given component in a sample was calculated using these calibration curves.

### Comparative Example A

In a 250 milliliter (mL) round bottom flask equipped with mechanical stirrer, thermometer, nitrogen inlet and reflux condenser, 18.5 gram (g) (0.124 mole) of phthalic anhydride and 9.99 g (0.073 mole) of zinc chloride were charged followed by 26.25 g (0.278 mole) phenol and 2.91 g (0.024 mole) chloro sulphonic acid, while maintaining the round bottom flask in nitrogen atmosphere at 50 to 60° C. The reaction mixture was then heated with stirring at 115° C. During the course of the reaction of 18 to 20 hours, the reaction mixture progressively turned from yellowish orange to brownish orange to deep brown while gaining viscosity. The reaction mixture was then treated with 250 mL hot water (50 to 90° C.). The treated mixture was stirred further for about 30 minutes at 80 to 85° C. The treated mixture was filtered while hot and washed with cold de-ionized water, then dried in an oven, resulting in a crude phenolphthalein material. The crude phenolphthalein material comprises 95 wt % phenolphthalein based on the total weight of the material. The molar yield based on phthalic anhydride was 90%.

### Example 1

In a 250 mL round bottom flask equipped with mechanical stirrer, thermometer, nitrogen inlet and reflux condenser, 18.5 g (0.124 mole) of phthalic anhydride and 9.99 g (0.073 mole) of zinc chloride were charged followed by 26.25 g (0.278 mole) phenol and 2.91 g (0.024 mole) chlorosulphonic acid, while maintaining the round bottom flask in nitrogen atmosphere at 50 to 60° C. The reaction mixture was then heated with stirring at 115° C. for 18 to 20 hours. During the course of the reaction of 18 to 20 hours, the reaction mass progressively turned from yellowish orange to brownish orange to deep brown while gaining viscosity. The reaction mass was cooled to room temperature and a solvent system containing a mixture of methanol and toluene (10:90, v:v) was added and stirred for 1 hour at reflux. The viscous mass became very free and the product was filtered. Hot water (50 to 90° C.) was added and then the reaction mixture was heated at 80 to 85° C. for 30 minutes and then cooled to room temperature. The resulting brownish yellow solid was dried in an oven at 100° C., over night. The solid comprised 98 wt % phenolphthalein based on the total weight of the solid as analyzed by HPLC. The molar yield based on phthalic anhydride was 85%.

### Comparative Example B

In a 500 mL four neck round bottom flask fitted with an overhead stirrer, a nitrogen gas inlet, thermowell, and a reflux condenser were placed 100.0 g (0.675 mole) of phthalic anhydride, 143.0 g (1.52 mole) of phenol, 55.20 g of zinc chloride (0.40 mole), and 15.73 g of chloro sulphonic acid (0.135 mole). A slow stream of nitrogen gas was continuously passed through the flask, and the reaction mixture was heated at 115° C. for 17 to 18 hours. The reaction mixture was allowed to cool to 50° C. then 900 mL of water was added to the reaction mixture followed by stirring for 2 hours at 85 to 90° C. The slurry formed was filtered out, washed with 100 mL water for 5 times until free from acid, and dried, resulting in crude phenolphthalein material. The yield of this process was 190.50 g (89 mol %) based on the moles of phthalic anhydride. The crude phenolphthalein material comprised less than or equal to 95 wt % phenolphthalein based on the total weight of the material.

### Example 2

The crude phenolphthalein prepared as described above in Comparative Example B was purified using the following procedure. 100.0 g of crude phenolphthalein prepared as described above in Comparative Example B and 400 ml of mixture of methanol and toluene (2:98, v:v) were placed in a 1000 mL four neck round bottom flask fitted with an overhead stirrer, a nitrogen gas inlet, thermowell, and a condenser to form a treatment slurry. The treatment slurry was stirred at reflux for 30 minutes and then cooled to 5 to 110° C., filtered, washed with 50 ml of the same solvent system, and dried, resulting in a solid material. The solid material comprised greater than or equal to 98 wt % of phenolphthalein based on the total weight of the solid material. The yield of this process was 94.0 g phenolphthalein (94.0 wt %).

### Examples 3-20 and Comparative Examples C-F

Crude phenolphthalein as described above in Comparative Example B was purified using the following procedure. 100.0 g of crude phenolphthalein was stirred with 400 ml of varying solvent systems as shown in Table 1 to form a treatment slurry. The treatment slurry was stirred at reflux for 30 minutes and then cooled to 5 to 23° C. and maintained for 60 minutes at 5 to 23° C. as indicated in Table 1, filtered, washed with 50 ml of the same solvent system at 5 to 23° C. as indicated in Table 1, and dried. The “recovery after drying” value is determined by the following formula: (weight of phenolphthalein recovered after solvent treatment and drying)/weight of crude phenolphthalein used. The recovery after drying value is a measure of the combined loss of phenolphthalein and impurity. Examples 3-20 all show a recovery after drying value of 70% or greater and a purity of 97% or more. In contrast the Comparative Examples C-F show either an unacceptable loss of phenolphthalein (as shown by the recovery after drying value), no substantial improvement in purity, or both. The purification conditions, recovery, and purity data are shown in Table 1.

TABLE 1

| | | | Volume ratio of solvent | Temperature of | Recovery after | Initial purity | Final purity |
|---|---|---|---|---|---|---|---|
| | Solvent 1 | Solvent 2 | 1 to solvent 2 | solvent treatment | drying (wt %) | (wt %) | (wt %) |
| 3 | Methanol | Toluene | 25:75 | 5° C. | 74.0 | 94.3 | 98.8 |
| 4 | Methanol | Toluene | 13:87 | 5° C. | 86.0 | 94.3 | 98.5 |
| 5 | Methanol | Toluene | 10:90 | 5° C. | 89.7 | 94.3 | 98.3 |
| 6 | Methanol | Toluene | 5:95 | 23° C. | 92.0 | 94.3 | 97.5 |
| 7 | Methanol | Toluene | 2:98 | 23° C. | 94.0 | 94.3 | 98.2 |
| 8 | Methanol | — | — | 5° C. | 70.0 | 94.3 | 98.7 |
| 9 | Ethyl acetate | Toluene | 25:75 | 10° C. | 83.5 | 94.6 | 98.3 |
| 10 | Methanol | Hexane | 25:75 | 10° C. | 80.5 | 94.6 | 98.2 |
| 11 | Dichloroethane | Acetone | 25:75 | 10° C. | 70.5 | 94.6 | 98.4 |
| 12 | Dichloroethane | Ethyl acetate | 25:75 | 10° C. | 83.2 | 94.6 | 98.3 |
| 13 | Ethyl acetate | — | — | 10° C. | 72.0 | 94.6 | 98.4 |
| 12 | Acetone | Toluene | 10:90 | 10° C. | 90.0 | 94.6 | 98.0 |
| 14 | Isopropanol | Toluene | 25:75 | 10° C. | 75.0 | 94.6 | 98.3 |
| 15 | Acetone | Hexane | 50:50 | 10° C. | 78.0 | 94.6 | 99.2 |
| C | — | Toluene | — | 5° C. | 100.0 | 94.3 | 94.3 |
| 16 | Dichloroethane | Toluene | 25:75 | 10° C. | 87.8 | 94.6 | 97.2 |
| D | Ethyl acetate | Hexane | 25:75 | 10° C. | 91.4 | 94.6 | 96.2 |
| E | Dichloroethane | Hexane | 25:75 | 10° C. | 90.0 | 94.6 | 95.8 |
| F | Methanol | Dichloroethane | 25:75 | 10° C. | 52.5 | 94.6 | 98.9 |
| 17 | Toluene | Phenol | 90:10 | 10° C. | 87.2 | 94.6 | 97.1 |
| 18 | Dichloroethane | Phenol | 90:10 | 10° C. | 86.8 | 94.6 | 97.3 |
| 19 | — | Dichloroethane | — | 10° C. | 92.0 | 94.6 | 97.6 |
| 20 | Chloroform | — | — | 10° C. | 70.0 | 94.6 | 97.5 |

### Examples 21-23

18.5 g (0.124 mole) of phthalic anhydride, 9.99 g (0.073 mole) of zinc chloride, 26.25 g (0.278 mole) phenol and 2.91 g (0.024 mole) chlorosulphonic acid were reacted under nitrogen at 110° C. for 18 hours. The reaction mass was cooled to 50 to 60° C. and a solvent system containing a mixture of methanol and toluene (10:90, v:v) was added in the amounts shown in Table 2. The resulting mixture is stirred at reflux temperature for 2 hours, and then cooled to 0 to 10° C. and maintained at 0 to 10° C. for 0.5 to 1.0 hour. In some examples the reaction mass was treated with the solvent system more than once as shown in Table 2. After filtering, the solid was washed with 300 mL of hot water (50 to 90° C., or more specifically, 70° C.), filtered and dried. Yields and purities are shown in Table 2. Yield is in mole percent based on the amount of phthalic anhydride. Purity is in wt % based on the total weight of the solid material. These examples show that using a solvent system comprising a polar organic solvent is an efficient method to obtain high purity phenolphthalein in a simple and efficient manner.

TABLE 2

| Example | Solvent System | Purity (wt %) | Yield (%) |
|---|---|---|---|
| 21 | 75 ml; 2 times | 97.77 | 85.5 |
| 22 | 75 ml; 1 time and | 97.88 | 84.2 |
| | 100 ml; 1 time | | |
| 23 | 150 ml; 1 time | 98.05 | 84.5 |

As used herein, the singular forms “a,” “an,” and “the” include plural referents unless the context clearly dictates otherwise. The endpoints of all ranges reciting the same characteristic or component are independently combinable and inclusive of the recited endpoint. “Crude phenolphthalein material” as used herein refers to a material that comprises less than or equal to 96 wt. % of phenolphthalein based on the total weight of the material. As used herein, the weight percentages of the isolated solid materials are based on the dry weight of the solid material. Suitable drying methods include air drying at ambient conditions or drying in an oven at elevated temperatures, for example, 50 to 100° C.

While typical embodiments have been set forth for the purpose of illustration, the foregoing descriptions should not be deemed to be a limitation on the scope herein. Accordingly, various modifications, adaptations, and alternatives can occur to one skilled in the art without departing from the spirit and scope herein.

1. A method of producing a phenolphthalein, comprising:
reacting a phthalic anhydride and a phenol in the presence of a catalyst and a promoter to form a reaction mixture comprising phenolphthalein;
treating the reaction mixture with a first solvent system to form a slurry, wherein the first solvent system comprises a first polar organic solvent;
filtering the slurry to obtain a first solid material;
washing the first solid material with water to obtain a second solid material, wherein the water is at a temperature of 25° C. to 90° C.;
wherein the dried second solid material comprises a phenolphthalein of formula (I) 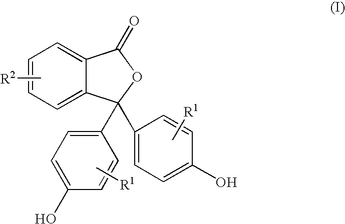
wherein R¹ is independently selected from the group consisting of a hydrogen and a hydrocarbyl group; R² is selected from the group consisting of a hydrogen, a hydrocarbyl group, and a halogen; and
wherein the second solid material comprises greater than or equal to 97 weight percent of phenolphthalein based on the total weight of the dried second solid material.

2. The method of claim 1, wherein the first polar organic solvent is selected from the group consisting of methanol, ethyl acetate, acetone, phenol, chloroform, and combinations of two or more of the foregoing.

3. The method of claim 1, wherein the first solvent system further comprises a non-polar solvent.

4. The method of claim 3, wherein the non-polar solvent is selected from the group consisting of toluene, hexane, cyclohexane, pentane, 1,2-dichloroethane, xylene, cumene, benzene, and combinations of two or more of the foregoing.

5. The method of claim 3, wherein the volume ratio of the polar solvent to the non-polar solvent is 5:95 to 50:50.

6. The method of claim 3, wherein the polar organic solvent is methanol and the non-polar solvent is toluene; and wherein the volume ratio of methanol to toluene is 2:98 to 25:75.

7. The method of claim 1, wherein the second solid material comprises greater than or equal to 98 weight percent of phenolphthalein.

8. The method of claim 1, wherein the method has a phenolphthalein molar yield of greater than or equal to 70 percent based on the molar amount of phthalic anhydride.

9. The method of claim 1, wherein the method has a phenolphthalein molar yield of greater than or equal to 80 percent based on the molar amount of phthalic anhydride.

10. The method of claim 1, further comprising:
dissolving the second solid material in a second solvent system to obtain a first solution, wherein the second solvent system comprises a second polar organic solvent;
crystallizing a third solid material from the second solution; and
isolating the third solid material;
wherein the isolated third solid material comprises greater than or equal to 98 weight percent of phenolphthalein based on the total weight of the isolated third solid material.

11. The method of claim 1, further comprising:
dissolving the second solid material in a second solvent system to obtain a first solution, wherein the second solvent system comprises a second polar organic solvent;
treating the first solution with a solid adsorbent to provide a first treatment mixture;
filtering the first treatment mixture to obtain a second solution;
crystallizing a third solid material from the second solution; and
isolating the third solid material;
wherein the third solid material comprises greater than or equal to 98 weight percent of phenolphthalein based on the total weight of the third solid material.

12. The method of claim 11, wherein the solid adsorbent is an activated carbon.

13. The method of claim 11, wherein the third solid material comprises greater than or equal to 99.6 weight percent of phenolphthalein based on the total weight of the third solid material.

14. A method of producing a phenolphthalein, comprising:
reacting a phthalic anhydride and a phenol in the presence of a metal halogenate catalyst and an acid promoter to form a reaction mixture comprising phenolphthalein;
treating the reaction mixture with a first solvent system to form a slurry, wherein the first solvent system comprises a polar and non-polar solvent;
filtering the slurry to obtain a first solid material;
washing the first solid material with water to obtain a second solid material,
wherein the water is at a temperature of 25° C. to 90° C.;
wherein the second solid material comprises a phenolphthalein of formula (I) 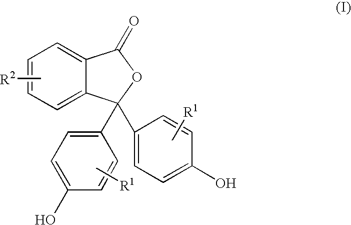
wherein R¹ is independently selected from the group consisting of a hydrogen and a hydrocarbyl group; R² is selected from the group consisting of a hydrogen, a hydrocarbyl group, and a halogen; and
wherein the second solid material comprises greater than or equal to 97 weight percent of phenolphthalein based on the total weight of the second solid material.

15. The method of claim 14, wherein the polar solvent is selected from the group consisting of methanol, ethanol, isopropanol, propanol, chloroform, acetone, ethyl acetate, phenol, and combinations of two or more of the foregoing.

16. The method of claim 14, wherein the non-polar solvent is selected from the group consisting of aromatic hydrocarbons having 6 to 14 carbons, aliphatic hydrocarbons having 5 to 8 carbons, non-polar chlorinated hydrocarbons, and combinations of two or more of the foregoing.

17. The method of claim 14, wherein the non-polar solvent is selected from the group consisting of toluene, hexane, cyclohexane, pentane, 1,2-dichloroethane, xylene, cumene, benzene, carbon tetrachloride, and combinations of two or more of the foregoing.

18. The method of claim 14, wherein the polar solvent is methanol and the non-polar solvent is toluene.

19. The method of claim 14, wherein the second solid material comprises greater than or equal to 98 weight percent of phenolphthalein based on the total weight of the third solid material.

20. The method of claim 14, wherein the method has a phenolphthalein molar yield of greater than or equal to 70 percent based on phthalic anhydride.

21. The method of claim 14, further comprising:
dissolving the second solid material in a second solvent system to obtain a first solution, wherein the second solvent system comprises a second polar organic solvent;
crystallizing a third solid material from the first solution; and
isolating the third solid material;
wherein the third solid material comprises greater than or equal to 98 weight percent of phenolphthalein based on the total weight of the third solid material.

22. The method of claim 14, further comprising:
dissolving the second solid material in a second solvent system to obtain a first solution, wherein the second solvent system comprises a second polar organic solvent;
treating the first solution with a solid adsorbent to provide a first treatment mixture;
filtering the first treatment mixture to obtain a second solution;
crystallizing a third solid material from the second solution; and
isolating the third solid material;
wherein the third solid material comprises greater than or equal to 98 weight percent of phenolphthalein based on the total weight of the third solid material.

23. The method of claim 22, wherein the solid adsorbent is an activated carbon.

24. The method of claim 22, wherein the third solid material comprises greater than or equal to 99.6 weight percent of phenolphthalein based on the total weight of the third solid material.

25. A method of purifying a phenolphthalein, comprising:
treating a crude phenolphthalein material with a first solvent system to form a slurry, wherein the first solvent system comprises a first polar organic solvent;
filtering the slurry to obtain a first solid material;
washing the first solid material with water to obtain a second solid material, wherein the water is at a temperature of 25° C. to 90° C.;
wherein the second solid material comprises a phenolphthalein of formula (I) 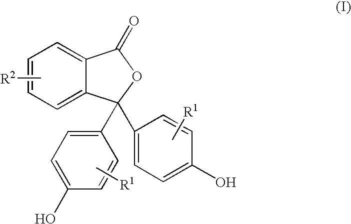
wherein R¹ is independently selected from the group consisting of a hydrogen and a hydrocarbyl group; R² is selected from the group consisting of a hydrogen, a hydrocarbyl group, and a halogen; and
wherein the second solid material comprises greater than or equal to 97 weight percent of phenolphthalein based on the total weight of the second solid material.

26. The method of claim 25, further comprising:
dissolving the second solid material in a second solvent system to obtain a first solution, wherein the second solvent system comprises a second polar organic solvent;
crystallizing a third solid material from the first solution; and
isolating the third solid material;
wherein the third solid material comprises greater than or equal to 98 weight percent of phenolphthalein based on the total weight of the isolated third solid material.

27. The method of claim 25, wherein the first solvent system further comprises a non-polar organic solvent.

28. The method of claim 25, further comprising:
dissolving the second solid material in a second solvent system to obtain a first solution, wherein the second solvent system comprises a second polar organic solvent;
treating the first solution with a solid adsorbent to provide a first treatment mixture;
filtering the first treatment mixture to obtain a second solution:
crystallizing a third solid material from the second solution;
isolating the third solid material;
wherein the third solid material comprises greater than or equal to 98 weight percent of phenolphthalein based on the total weight of the isolated third solid material.

29. A method of producing a phenolphthalein, comprising:
reacting a phthalic anhydride and a phenol in the presence of zinc chloride and chloro sulphonic acid to form a reaction mixture comprising phenolphthalein;
treating the reaction mixture with a first solvent system to form a slurry, wherein the first solvent system comprises a methanol and toluene;
filtering the slurry to obtain a first solid material;
washing the first solid material with water to obtain a second solid material, wherein the water is at a temperature of 25° C. to 90° C.;
wherein the second solid material comprises a phenolphthalein of formula (I) 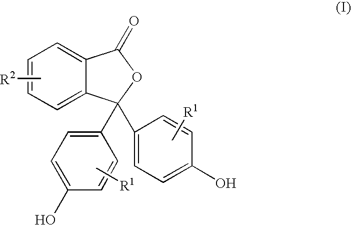
wherein R¹ is independently selected from the group consisting of a hydrogen and a hydrocarbyl group; R¹ is selected from the group consisting of a hydrogen, a hydrocarbyl group, and a halogen; and
wherein the second solid material comprises greater than or equal to 97 weight percent of phenolphthalein based on the total weight of the second solid material.

30. The method of claim 29, further comprising:
dissolving the second solid material in a second solvent system to obtain a first solution, wherein the second solvent system comprises methanol;
crystallizing a third solid material from the first solution; and
isolating the third solid material;
wherein the third solid material comprises greater than or equal to 98 weight percent of phenolphthalein based on the total weight of the isolated third solid material.
